(19) United States
(12) Patent Application Publication
(10) Pub. No.: US 2009/0182285 A1
(43) Pub. Date: Jul. 16, 2009

(54) Syringe Using In Fat Transplantation

(75) Inventors: HeeYoung LEE, Kunsan (KR); JunSeok Lee, Busan (KR)

Correspondence Address: SUMMA, ADDITON & ASHE, P.A., 11610 NORTH COMMUNITY HOUSE ROAD, SUITE 200, CHARLOTTE, NC, 28277, US

(73) Assignee: MEDIKAN CO., LTD., Busan (KR)

(21) Appl. No.: 12/349,623

(22) Filed: Jan. 7, 2009

(30) Foreign Application Priority Data
Jul. 11, 2006 (KR) KR10-2006-0064946
Jul. 10, 2007 (KR) PCT/KR07/03343

(51) Int. Cl. A61M 5/315

(52) U.S. Cl. 604/228

(57) ABSTRACT

The present invention relates to a syringe used in fat transplantation comprising: a cylinder having a coupling part formed at one side thereof such that a cannula, a needle or a cap is detachably coupled, an open part formed at the other end, and a flange formed at an outer periphery of the open part; a piston head installed in the cylinder; and a plunger for reciprocating the piston head, characterized in that the flange is formed separately from the cylinder, and a flange attachment structure is formed between the flange and the cylinder to detach the flange from the cylinder, wherein the flange attachment structure is comprised of a hooking projection projecting from an outer periphery of the cylinder, an insertion groove formed in an inner periphery of the flange such that the hooking projection is inserted, and a hooking groove integrally formed with the insertion groove such that the hooking projection is hooked.

### CROSS-REFERENCE TO PRIORITY APPLICATION

This application hereby claims the benefit of the commonly assigned Korean Application No. 10-2006-0064946 (filed Jul. 11, 2006, in the Korean Patent Office) and the commonly assigned International Patent Application No. PCT/KR2007/003343 (filed Jul. 10, 2007, in the Korean Patent Office), both of which are hereby incorporated by reference in their entirety.

### FIELD OF THE INVENTION

The present invention relates to a syringe used in fat transplantation, and more particularly, to a syringe used in fat transplantation capable of doubling as a fat tissue storage vessel and minimizing a space occupied in a storage chamber to thereby increase storage efficiency and facilitate fat transplantation.

### BACKGROUND

Fat transplantation, which has abruptly increased in popularity and become more widely used in recent times, is an operation in which fat from a part of a human body collected through fat suction is transplanted to another part of the human body where the fat is needed or desired. The fat remaining after the fat transplantation is frozen and stored in a syringe without being contained in a separate storage vessel, and then the fat may be thawed to be re-injected. As the number of operations has increased, the need for a freezing device for storing fat has also abruptly increased. In addition, since storage vessels for the fat of numerous patients may be stored in an overlapping manner, the probability of cross contamination may be increased, even in the frozen state. In order to reduce the probability of cross contamination, additional efforts and processes related to dual packaging are needed.

In a conventional syringe used for fat transplantation, when the fat remaining after the fat suction, or the fat remaining in a cylinder after the fat transplantation, is stored, a cannula and a needle of the syringe are separated from a coupling part of the cylinder after the fat suction and fat transplantation, the coupling part is sealed by a cap, a plunger coupled to a piston head in the cylinder is separated therefrom to reduce an occupied space, and then the fat is stored in the freezing device or in a liquid nitrogen tank.

Generally, a flange is integrally formed with a cylinder of the conventional syringe to improve grip of the cylinder. However, since the flange projects outward from the cylinder to some extent, it is difficult to store cylinder in close proximity to each other, and thus a large space may be occupied and storage efficiency may be decreased. In particular, while cryogenic storage, such as liquid nitrogen, is widely used nowadays due to its advantages, as consumption of liquid nitrogen tanks also increases and a large tank capacity increases a manufacturing cost, storage and maintenance cost may also be increased several times more than a conventional freezing device.

In addition, since the syringe is stored in liquid nitrogen, the cylinder, the piston and the cap of the syringe may abruptly shrink to thereby decrease sealing performance, and the probability of cross contamination due to intrusion of the liquid nitrogen may increase.

### SUMMARY OF THE INVENTION

In order to solve the above problems, it is an object of the present invention to provide a syringe used in fat transplantation capable of doubling as a fat tissue storage vessel, minimizing a space occupied in a storage chamber to increase storage efficiency, and reducing storage and maintenance cost. In addition, it is possible to improve sealing performance of the vessel to secure against cross contamination. Further, since a plunger can be readily moved forward with low power and rapidly moved backward, fat suction and transplantation can be very conveniently performed.

The foregoing and/or other objects of the present invention may be achieved by providing a syringe used in fat transplantation comprising: a cylinder having a coupling part formed at one side thereof such that a cannula, a needle or a cap is detachably coupled, an open part formed at the other end, and a flange formed at an outer periphery of the open part; a piston head installed in the cylinder; and a plunger for reciprocating the piston head, characterized in that the flange is formed separately from the cylinder, and a flange attachment structure is formed between the flange and the cylinder to detach the flange from the cylinder.

In addition, the flange attachment structure may comprise a hooking projection projecting from an outer periphery of the cylinder, an insertion groove formed in an inner periphery of the flange such that the hooking projection is inserted, and a hooking groove integrally formed with the insertion groove such that the hooking projection is hooked.

Further, the syringe used in fat transplantation may further comprise a first sealing member for sealing the open part of the cylinder.

Furthermore, the syringe used in fat transplantation may further comprise a second sealing member having an insertion groove and a hooking groove corresponding to the insertion groove and the hooking groove of the flange, which are formed at an inner periphery of thereof.

In addition, in the coupling structure of the cylinder and the cap, the coupling part of the cylinder may be comprised of an inner tube and an outer tube, the cap detachably coupled to the coupling part may be comprised of an inner tube press-fitted between the inner and outer tubes of the coupling part and an outer tube inserted into an outer periphery of the outer tube of the coupling part, and a packing may be disposed between the outer tube of the cylinder and the outer tube of the cap.

Further, an outer periphery of the inner tube of the cylinder and an inner periphery of the inner tube of the cap press-fitted into the cylinder may be tapered.

Furthermore, threaded parts may be formed at an inner periphery of the outer tube of the cylinder and an outer periphery of the inner tube of the cap to be threadedly engaged with each other.

In addition, a spiral groove may be formed at an outer periphery of the plunger, and a locker having a guide projection inserted into the spiral groove may be installed at the flange. The locker may be slidably installed at the flange in a direction such that the guide projection is inserted into or separated from the spiral groove.

According to a syringe used in fat transplantation, a coupling part and an open part of a cylinder are sealed to use the syringe as a fat storage vessel. In addition, a flange can be removed from the cylinder to store the cylinders in close proximity in a freezing device or a liquid nitrogen tank, thereby increasing storage efficiency. As a result, it is possible to reduce maintenance cost when the liquid nitrogen tank is used.

Further, since a sealing structure of the coupling part and the open part of the cylinder has a dual structure, sealing performance can be improved, and cross contamination during storage of the cylinder due to leakage of fat or intrusion of liquid nitrogen can be prevented.

Furthermore, since a plunger can be reciprocated by selecting rotation of the plunger using a spiral groove and a locker of the plunger or reciprocal movement of the plunger, it is possible to conveniently perform fat suction and transplantation. In particular, when a high pressure is applied into the cylinder during fat transplantation, the plunger is rotated and moved forward with the locker being hooked by the spiral groove to perform the fat transplantation with low power. In addition, when a user intends to rapidly pull back the plunger or separate the plunger from the cylinder, the locker can be rapidly pulled back from the spiral groove by releasing the locker, thereby providing user convenience.

The foregoing and other objects and advantages of the invention and the manner in which the same are accomplished will become clearer based on the following detailed description taken in conjunction with the accompanying drawings.

### BRIEF DESCRIPTION OF THE DRAWINGS

FIG. 1 is a cross-sectional view of a syringe used in fat transplantation in accordance with an exemplary embodiment of the present invention;

FIG. 2 is an exploded perspective view of the syringe used in fat transplantation in accordance with an exemplary embodiment of the present invention;

FIGS. 3 and 4 are cross-sectional views showing the attachment operation of a flange of a syringe in accordance with another exemplary embodiment of the present invention;

FIG. 5 is a cross-sectional view of the syringe used as a storage vessel in accordance with the present invention;

FIG. 6 is an exploded perspective view of the syringe used as a storage vessel in accordance with the present invention; and

FIGS. 7 and 8 are views of a plunger and a locker in the syringe in accordance with the present invention.

### DETAILED DESCRIPTION

Exemplary embodiments of a syringe used in fat transplantation in accordance with the present invention will now be described in detail with reference to the accompanying drawings.

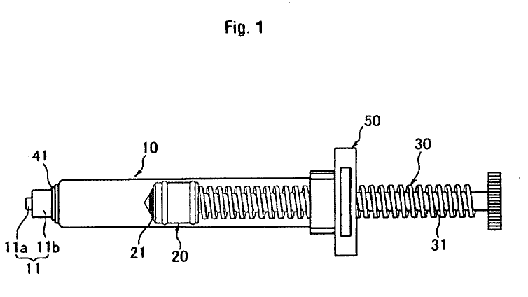

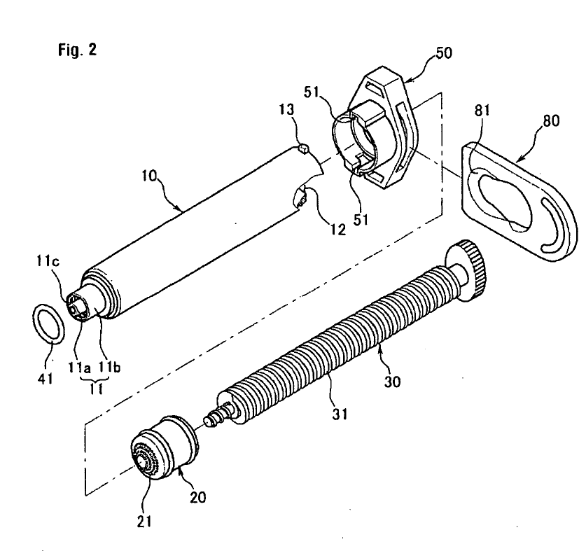

FIGS. 1 and 2 are a cross-sectional view and an exploded perspective view of a syringe used in fat transplantation in accordance with an exemplary embodiment of the present invention. As shown in FIGS. 1 and 2, the syringe includes a cylinder 10, a piston head 20 installed in the cylinder 10, and a plunger 30 detachably coupled to the piston head 20 to reciprocate the piston head 20 to thereby suck and transplant fat.

The cylinder 10 has a coupling part 11 formed at one side. A cannula and a needle (not shown) are detachably coupled to the coupling part 11 to suck and transplant fat. When the cannula and the needle are not used, a cap 40 is coupled to the coupling part 11 to seal the coupling part 11.

In addition, the cylinder 10 has an open part 12 formed at the other side. A flange 50 projects from an outer periphery of the cylinder 10 adjacent to the open part 12 to readily grip the cylinder 10 and to prevent a user's hand from sliding. The flange 50 is formed separately from the cylinder 10, and thus, the flange 50 can be detachably coupled to the cylinder 10 by a flange attachment structure formed between the flange 50 and the cylinder 10.

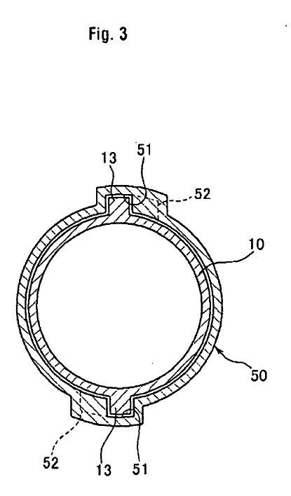

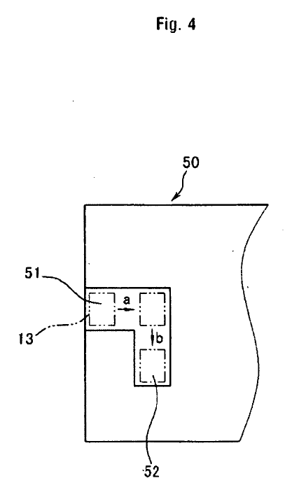

As shown in FIGS. 2, 3 and 4, the flange attachment structure is comprised of a hooking projection 13 projecting from an outer periphery of the cylinder 10, an insertion groove 51 formed in the flange 50 such that the hooking projection 13 is inserted, and a hooking groove 52 to which the hooking projection 13 is hooked. The insertion groove 51 is formed in the cylinder 10 in a longitudinal direction thereof, and the hooking groove 52 is integrally formed with an end of the insertion groove 51 to form a right angle. Therefore, as shown in FIG. 4, the hooking projection 13 is inserted into the insertion groove 51 (an arrow direction a) and rotated toward the hooking groove 52 (an arrow direction b) to couple the flange 50 to the cylinder 10. Alternatively, the flange 50 can be separated from the cylinder 10 through an operation in reverse to that described above. The flange attachment structure may be formed at a single position or may be formed at two positions at 180° intervals as in the embodiment to stably perform the attachment operation.

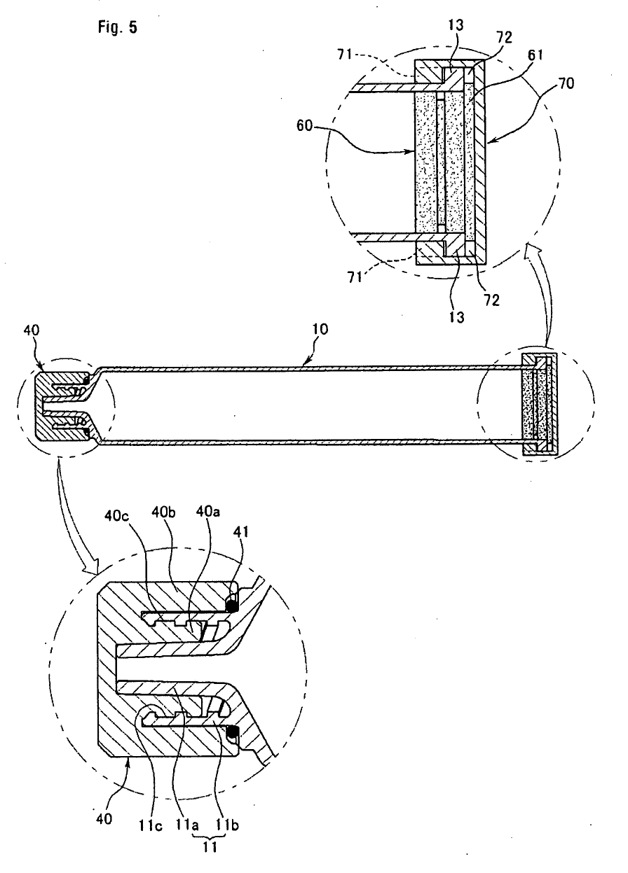

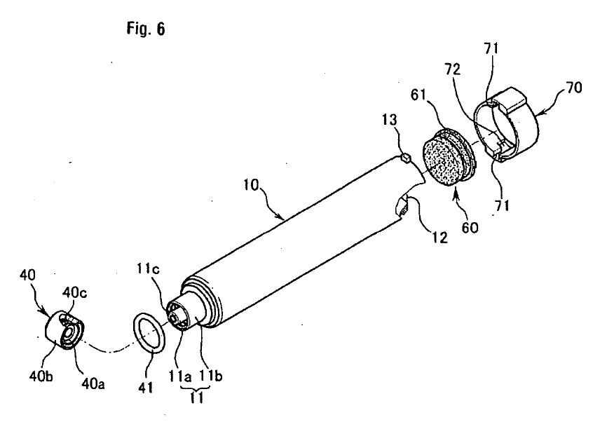

In addition, as shown in FIGS. 5 and 6, the syringe of the present invention further includes a first sealing member 60 for sealing the open part 12 of the cylinder 10. An outer periphery of the first sealing member 60 is closely adhered to an inner periphery of the cylinder 10 to seal the open part 12 of the cylinder 10. The first sealing member 60 may be formed of a silicon or rubber material. The first sealing member 60 has a small hooking part 61 formed at an end thereof and having a diameter larger than an inner diameter and equal to or smaller than an outer diameter of the cylinder 10, to prevent the first sealing member 60 from being fully inserted into the cylinder 10.

The syringe of the present invention further includes a second sealing member 70 for sealing the open part 12 of the cylinder 10. The second sealing member 70 has an insertion groove 71 and a hooking groove 72 corresponding to the insertion groove 51 and the hooking groove 52 of the flange 50 to be detachably coupled to the cylinder 10, similar to the flange 50.

In addition, as shown in FIG. 5, an inner periphery of the second sealing member 70 is adhered to an exposed surface of the hooking part 61 of the first sealing member 60 when the second sealing member 70 is coupled to the cylinder 10, to seal the open part 12 of the cylinder 10 in a dual sealing manner, i.e., sealing by adhesion between the inner periphery of the cylinder 10 and the first sealing member 60 and sealing by adhesion between the first sealing member 60 and the second sealing member 70.

Meanwhile, in the coupling structure of the cylinder 10 and the cap 40, the coupling part 11 of the cylinder 10 is comprised of an inner tube 11a and an outer tube 11b, the cap 40 detachably coupled to the coupling part 11 is comprised of an inner tube 40a press-fitted between the inner and outer tubes 11a and 11b and an outer tube 40b inserted into an outer periphery of the outer tube 11b, and a packing 41 is disposed between the outer tube 11b of the cylinder 10 and the outer tube 40b of the cap 40.

An outer periphery of the inner tube 11a of the cylinder 10 and an inner periphery of the inner tube 40a of the cap 40 may be tapered such that they are closely adhered to each other when the cap 40 is coupled to the cylinder 10, thereby sealing the cylinder 10 together with the packing 41 in a dual sealing manner. In addition, threaded parts 11c and 40c are formed at the inner periphery of the outer tube 11b of the cylinder 10 and the outer periphery of the inner tube 40a of the cap 40 to be detached from each other in a threaded manner.

In addition, as shown in FIGS. 1 and 2, a spiral groove 31 is formed in an outer periphery of the plunger 30, and a locker 80 having a guide projection 81 inserted into the spiral groove 31 is installed at the flange 50 such that the guide projection 81 is slidably inserted into or separated from the spiral groove 31.

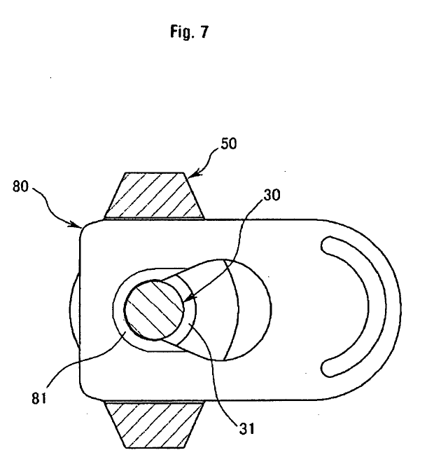

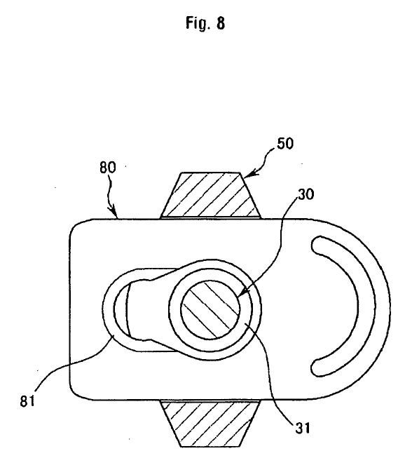

Therefore, as shown in FIG. 7, when the guide projection 81 of the locker 80 is inserted into the spiral groove 31 of the plunger 30, it is impossible to directly reciprocate the plunger 30, and it is possible to reciprocate the piston head 20 by rotating the plunger 30. In addition, as shown in FIG. 8, when the locker 80 is slid in a direction such that the guide projection 81 is separated from the spiral groove 31, the plunger 30 can be directly and straightly moved to reciprocate the piston head 20.

While the piston head 20 has a porous net 21 to separate free oil to perform a filter function during centrifugation, e.g., as disclosed in Korean Utility Model Registration No. 327374, but not limited thereto, a general piston head having no filter function may be employed.

As shown in FIG. 1, the cannula or the needle (not shown) is coupled to the coupling part 11 of the cylinder 10 to reciprocate the plunger 30, thereby performing fat suction from or fat transplantation to a human body. In addition, the syringe in accordance with the present invention can double as a storage vessel for storing the fat sucked from the human body, or storing the fat remaining in the cylinder 10 after the fat transplantation. That is, as shown in FIGS. 5 and 6, the cannula or the needle is separated from the coupling part 11 of the cylinder 10, the cap 40 is coupled to the cylinder 10, the piston head, the plunger 30 and the flange 50 are separated from the cylinder 10, and then the open part 12 of the cylinder 10 is sealed by the first and second sealing members 70.

When the flange 50 is separated from the cylinder 10, as shown in FIG. 4, the hooking projection 13 of the cylinder 10 is hooked by the hooking groove 52 of the flange 50 to rotate the flange 50 in a circumferential direction (a reverse direction of an arrow b) of the cylinder 10 to release the hooking projection 13 from the hooking groove 52 with the flange 50 being coupled to the cylinder 10. Then, when the flange 50 is pulled in a longitudinal direction (a reverse direction of an arrow a) of the cylinder 10, the hooking projection 13 is released through the insertion groove 51 to simply separate the flange 50.

As described above, since the flange 50 is fully separated from the cylinder 10, when the cylinder 10 is stored in the freezing device or the liquid nitrogen tank, the cylinders 10 can be closely adhered to each other and stored. As a result, since a larger number of cylinders 10 can be stored in a limited space, it is possible to increase the storage efficiency of liquid nitrogen tanks to thereby remarkably reduce maintenance cost.

In addition, as shown in FIG. 5, according to the coupling structure of the coupling part 11 of the cylinder 10 and the cap 40, the inner tube 11a and the outer tube 11b of the cylinder 10 are coupled to the inner tube 40a and the outer tube 40b of the cap 40 in a dual sealing manner, and the packing seals between the outer tube 11b of the cylinder 10 and the outer tube 40b of the cap 40. At the same time, the tapered outer periphery of the inner tube 11a of the cylinder 10 is closely adhered to the tapered inner periphery of the inner tube 40a of the cap 40 press-fitted into the inner tube 11a of the cylinder 10 to seal the coupling part 11 in a dual sealing manner. In the sealing structure of the open part 12 of the cylinder 10, since a space between the inner periphery of the cylinder 10 and the first sealing member 60 is sealed and a space between the inner surface of the second sealing member 70 and one surface of the hooking part 61 of the first sealing member 60 is sealed, the open part 12 is sealed in a dual sealing manner. Therefore, since the sealing performance between the coupling part 11 and the open part 12 of the cylinder 10 is improved, it is possible to prevent cross contamination due to leakage of fat or intrusion of liquid nitrogen into the storage vessel.

Further, it is possible to conveniently perform the fat suction and fat transplantation using the spiral groove 31 installed at the plunger 30 and the locker 80. That is, as shown in FIGS. 1 and 7, since the piston head 20 can be reciprocated by rotating the plunger 30 in a state such that the guide projection 81 of the locker 80 is inserted into the spiral groove 31 of the plunger 30, when a high pressure is applied into the cylinder 10 to require high forward movement power of the plunger 30, the plunger 30 is rotated and moved forward to conveniently inject the fat into the human body. As shown in FIG. 8, when the locker 80 is slid in a direction such that the guide projection 81 is separated from the spiral groove 31, since the spiral groove 31 is released from the guide projection 81 to enable direct movement of the plunger 30 in a straight direction to thereby reciprocate the piston head 20, it is possible to rapidly pull back the piston head 20 when the plunger 30 is pulled back or separated from the cylinder 10.

While this invention has been described with reference to exemplary embodiments thereof, it will be clear to those of ordinary skill in the art to which the invention pertains that various modifications may be made to the described embodiments without departing from the spirit and scope of the invention as defined in the appended claims and their equivalents.

As can be seen from the foregoing, a syringe used in fat transplantation in accordance with the present invention can double as a storage vessel in addition to fat suction and fat transplantation and minimize a space occupied in a storage chamber, thereby increasing storage efficiency and reducing storage and maintenance cost. In addition, it is possible to improve sealing performance of the storage vessel to secure against cross contamination. Further, since the plunger can be moved with low power and rapidly pulled back, the fat suction and the fat transplantation can be conveniently performed.

1. A syringe used in fat transplantation comprising:
a cylinder having a coupling part formed at one side thereof such that a cannula, a needle or a cap is detachably coupled thereto, an open part formed at the other end, and a flange formed at an outer periphery of the open part;
a piston head installed in the cylinder;
and a plunger for reciprocating the piston head;
wherein the flange is formed separately from the cylinder, and a flange attachment structure is formed between the flange and the cylinder to detach the flange from the cylinder.

2. The syringe used in fat transplantation according to claim 1, wherein the flange attachment structure comprises:
a hooking projection projecting from an outer periphery of the cylinder;
an insertion groove formed in an inner periphery of the flange such that the hooking projection is inserted; and
a hooking groove integrally formed with the insertion groove such that the hooking projection is hooked.

3. The syringe used in fat transplantation according to claim 2, further comprising a first sealing member for sealing the open part of the cylinder.

4. The syringe used in fat transplantation according to claim 3, further comprising a second sealing member having an insertion groove and a hooking groove corresponding to the insertion groove and the hooking groove of the flange, which are formed at an inner periphery of thereof.

5. The syringe used in fat transplantation according to claim 1, wherein, in the coupling structure of the cylinder and the cap, the coupling part of the cylinder is comprised of an inner tube and an outer tube, the cap detachably coupled to the coupling part is comprised of an inner tube press-fitted between the inner and outer tubes of the coupling part and an outer tube inserted into an outer periphery of the outer tube of the coupling part, and a packing is disposed between the outer tube of the cylinder and the outer tube of the cap.

6. The syringe used in fat transplantation according to claim 5, wherein an outer periphery of the inner tube of the cylinder and an inner periphery of the inner tube of the cap press-fitted into the cylinder are tapered.

7. The syringe used in fat transplantation according to claim 6, wherein threaded parts are formed at an inner periphery of the outer tube of the cylinder and an outer periphery of the inner tube of the cap to be threadedly engaged with each other.

8. The syringe used in fat transplantation according to claim 1, wherein a spiral groove is formed at an outer periphery of the plunger, and a locker having a guide projection inserted into the spiral groove is installed at the flange, wherein the locker is slidably installed at the flange in a direction such that the guide projection is inserted into or separated from the spiral groove.
